(19) United States
(12) Patent Application Publication
(10) Pub. No.: US 2024/0285875 A1
(43) Pub. Date: Aug. 29, 2024

(54) ATOMIZING APPARATUS

(71) Applicant: Murata Manufacturing Co., Ltd., Kyoto (JP)

(72) Inventors: Yohei KAWASAKI, Kyoto (JP); Kenghua HSING, Kyoto (JP); Hiromu INOUE, Kyoto (JP)

(21) Appl. No.: 18/654,333

(22) Filed: May 3, 2024

Related U.S. Application Data
(63) Continuation of application No. PCT/JP2022/040227, filed on Oct. 27, 2022.

(30) Foreign Application Priority Data
Nov. 10, 2021 (JP) 2021-183033

(51) Int. Cl. A61M 11/02; A61M 11/04

(52) U.S. Cl. CPC A61M 11/02; A61M 11/042

(57) ABSTRACT

An atomizing apparatus having a top surface and a bottom surface includes a first tubular body, a second tubular body, pumps, and an atomizing portion. The pumps are disposed in the first tubular body and have an intake hole and a discharge hole. The atomizing portion is configured to atomize a liquid supplied to the housing by a gas discharged through the discharge hole of one of the pumps. The first tubular body has a first cavity and a first communication hole that is in communication with the second tubular body. The second tubular body has a second cavity and a through-hole. The through-hole is in communication with the first communication hole. The atomizing portion is disposed at the through-hole. The first cavity is disposed at a position closer than the through-hole to the top surface.

### CROSS REFERENCE TO RELATED APPLICATION

This is a continuation of International Application No. PCT/JP2022/040227 filed on Oct. 27, 2022 which claims priority from Japanese Patent Application No. 2021-183033 filed on Nov. 10, 2021. The contents of these applications are incorporated herein by reference in their entireties.

### BACKGROUND OF THE DISCLOSURE

### Field of the Disclosure

The present disclosure relates to an atomizing apparatus that atomizes and discharges a liquid.

### Description of the Related Art

Patent Document 1 discloses a nebulizer. The nebulizer of Patent Document 1 has a nozzle hole disposed at the upper end of a casing body and configured to discharge compressed air. The compressed air is discharged upward from the casing body.

The nebulizer of Patent Document 1 has an aerosol outlet through which a liquid to be atomized is discharged, and the aerosol outlet is also disposed at the upper end of the casing body. The liquid is discharged from the aerosol outlet to the upper end of the casing body, and the compressed air discharged from the casing body atomizes the liquid.

Patent Document 1: Japanese Unexamined Patent Application Publication No. 2013-132471

### BRIEF SUMMARY OF THE DISCLOSURE

In the structure disclosed in Patent Document 1, the liquid may enter the casing body through the nozzle hole. If this occurs, in the case where a pump for generating compressed air is disposed inside the casing body, the liquid may enter the pump and cause malfunction.

Accordingly, a possible benefit of the present disclosure is to provide an atomizing apparatus that can reduce the occurrence of malfunction or failure due to unexpected liquid flow.

According to the present disclosure, an atomizing apparatus includes a housing having opposing first and second ends in a first direction of the housing, a first tubular body and a second tubular body formed in the housing, a pump, and an atomizing portion. The pump is disposed in the first tubular body and has an intake hole and a discharge hole, and the pump is configured to take a gas through the intake hole and discharge the gas through the discharge hole. The atomizing portion is configured to atomize a liquid supplied to the housing by using the gas discharged through the discharge hole of the pump. The first tubular body has a first cavity and a first communication hole that is in communication with the second tubular body. The second tubular body has a second cavity and a second communication hole. The atomizing portion is disposed at a position adjacent to the second communication hole. The first cavity is disposed at a position closer than the first communication hole to the first end. The pump is disposed between the first communication hole and the first cavity in the first direction. The discharge hole of the pump is disposed at a position closer than the first communication hole to the first end in the first direction.

In this configuration, the liquid that has flowed from the atomizing portion into the first tubular body is stored at a bottom surface (i.e., an inside surface of the bottom wall of the housing), which is positioned at the second end of the first tubular body. Accordingly, the liquid having flowed into the first tubular body does not reach the pump easily.

According to the present disclosure, the atomizing apparatus can reduce the occurrence of malfunction or failure even if an unexpected flow of the liquid occurs.

### BRIEF DESCRIPTION OF THE SEVERAL VIEWS OF THE DRAWINGS

FIG. 1 is a perspective view illustrating an exterior of an atomizing apparatus according to a first embodiment.

FIG. 2A is a front view illustrating part of the atomizing apparatus of the first embodiment, FIG. 2B is a cross-sectional side view illustrating part of a structure of the atomizing apparatus of the first embodiment, and FIG. 2C is a rear view illustrating part of the atomizing apparatus of the first embodiment.

FIG. 3 is an enlarged view illustrating a part including an atomizing portion in the cross-sectional side view of FIG. 2B.

FIG. 4 is a cross-sectional side view illustrating the atomizing apparatus of the first embodiment when the atomizing apparatus is in normal operation.

FIG. 5 is an enlarged cross-sectional view illustrating an example state in which a liquid is stored in a recess.

FIG. 6 is a view illustrating the atomizing apparatus being turned over on its side.

FIG. 7 is a cross-sectional side view illustrating part of a structure of an atomizing apparatus according to a second embodiment.

FIG. 8 is a cross-sectional view illustrating part of a structure of an atomizing apparatus according to an example derived from the second embodiment.

FIG. 9 is a cross-sectional side view illustrating part of a structure of an atomizing apparatus according to a third embodiment.

FIG. 10 is a cross-sectional view illustrating some flow paths in the atomizing apparatus of the third embodiment when the fluid paths are viewed in plan.

FIG. 11 is another cross-sectional side view schematically illustrating gas flow in the atomizing apparatus of the third embodiment.

### DETAILED DESCRIPTION OF THE DISCLOSURE

### First Embodiment

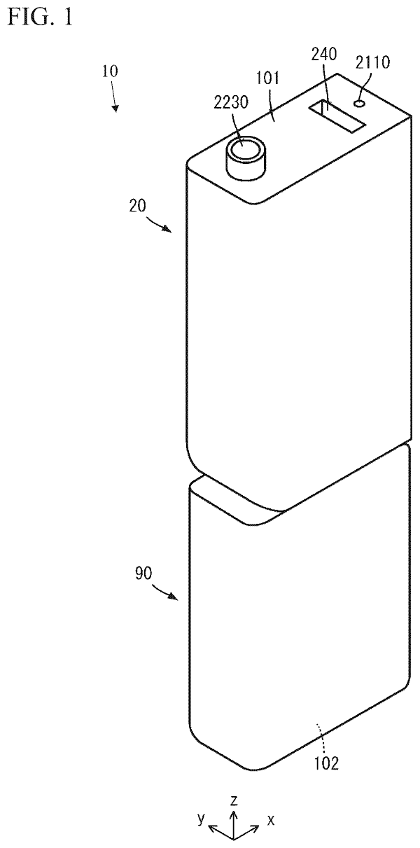

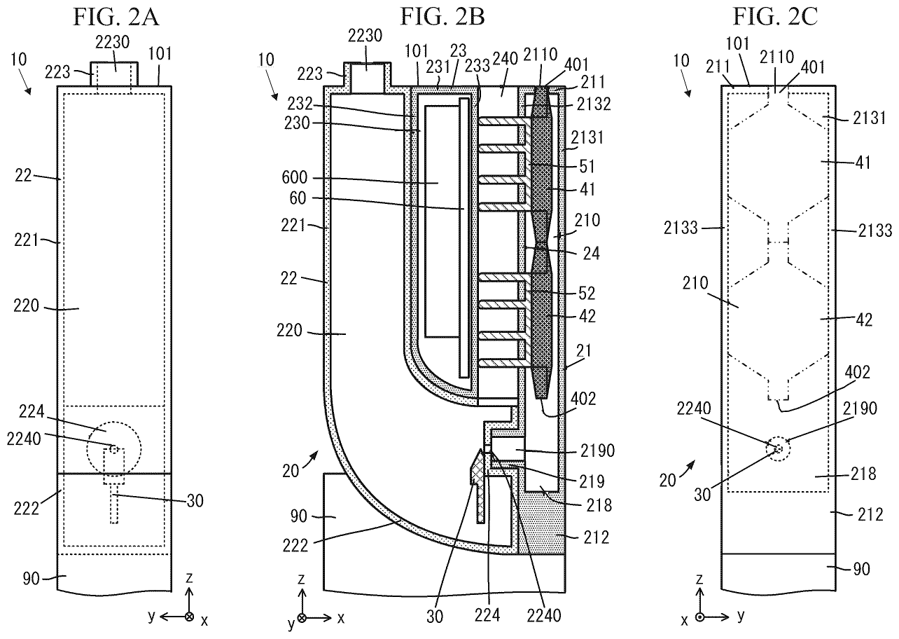

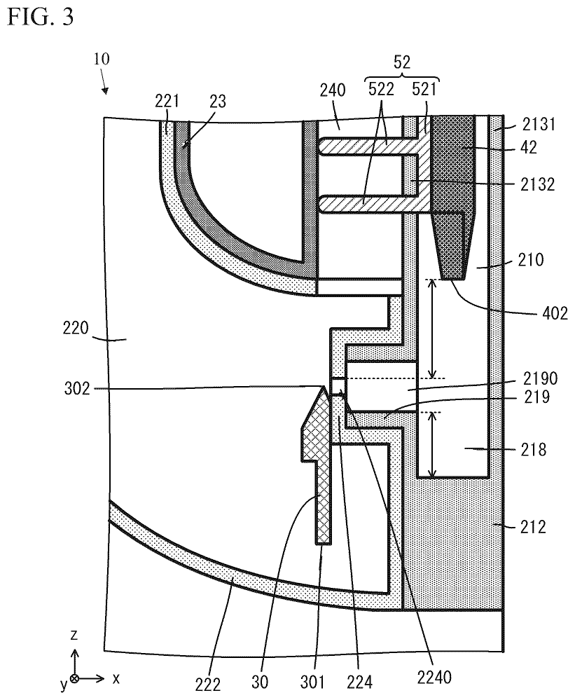

An atomizing apparatus according to a first embodiment of the present disclosure will be described with reference to the drawings. FIG. 1 is a perspective view illustrating an exterior of the atomizing apparatus of the first embodiment. FIG. 2A is a front view illustrating part of the atomizing apparatus of the first embodiment, FIG. 2B is a cross-sectional side view illustrating part of a structure of the atomizing apparatus of the first embodiment, and FIG. 2C is a rear view illustrating part of the atomizing apparatus of the first embodiment. In FIGS. 2A and 2C, the front and rear sides are defined by way of example for description convenience. The cross-sectional side view of FIG. 2B is provided only for the sake of clarifying a positional relationship of elements of the atomizing apparatus, which may be different in some parts from the actual view. FIG. 3 is an enlarged view illustrating a part including an atomizing portion in the cross-sectional side view of FIG. 2B.

### (Exterior of Atomizing Apparatus 10)

As illustrated in FIG. 1, an atomizing apparatus 10 includes a main body 20 and an auxiliary housing 90. The main body 20 and the auxiliary housing 90 both have substantially rectangular parallelepiped shapes in which some edges are rounded. The term “substantially rectangular parallelepiped shape” means a rectangular parallelepiped shape with rounded edges. The shapes of the main body 20 and the auxiliary housing 90, however, are not limited to the above.

The main body 20 and the auxiliary housing 90 are disposed side-by-side and connected to each other in the z-axis direction (i.e., the first direction of the atomizing apparatus 10). As viewed in plan (i.e., as viewed in the first direction), the main body 20 and the auxiliary housing 90 have substantially rectangular shapes extending in the x-axis direction (i.e., in the second direction of the atomizing apparatus 10) and also in the y-axis direction (i.e., in the third direction of the atomizing apparatus 10). The shapes of the main body 20 and the auxiliary housing 90 are substantially the same. The main body 20 and the auxiliary housing 90 overlap each other as viewed in plan.

The main body 20 and the auxiliary housing 90 constitute the housing of the atomizing apparatus 10. A top surface 101 of the housing is an end surface of the main body 20 in the z-axis direction, and a bottom surface 102 of the housing is an end surface of the auxiliary housing 90 in the z-axis direction. The top surface 101 corresponds to the “first end” of the housing of the present disclosure, and the bottom surface 102 corresponds to the “second end” of the housing of the present disclosure. In other words, the atomizing apparatus 10 is placed, in normal operation, such that the main body 20 comes above the auxiliary housing 90 or the auxiliary housing 90 comes below the main body 20.

A cavity 2110 (first cavity), a cavity 2230 (second cavity), and a cavity 240 (third cavity) are formed at the top surface 101 of the main body 20. The cavity 2110 and the cavity 240 are intake ports of a gas for atomizing a liquid, which will be described in detail later. The cavity 2230 is a discharge port for discharging the atomized liquid.

### (Specific Internal Structure of Atomizing Apparatus 10)

As illustrated in FIGS. 2A, 2B, 2C, and FIG. 3, the main body 20 of the atomizing apparatus 10 includes a tubular body 21 (first tubular body), a tubular body 22 (second tubular body), and a tubular body 24 (third tubular body). The main body 20 also includes a bar member 30, pumps 41 and 42, and heat conducting members 51 and 52. The main body 20 further includes a driving circuitry case 23.

The tubular body 21, the tubular body 24, the driving circuitry case 23, and the tubular body 22 are disposed in this order in the x-axis direction.

### (Shape of Tubular Body 21)

The tubular body 21 includes a top wall 211, a bottom wall 212, a side wall 2131, another side wall 2132, and another pair of side walls 2133.

More specifically, the side wall 2131, the side wall 2132, and the side walls 2133 have respective wall surfaces extending in the z-axis direction. The side wall 2131 and the side wall 2132 are spaced from each other in the x-axis direction. The side walls 2133 are joined to the side wall 2131 and the side wall 2132.

The top wall 211 and the bottom wall 212 have wall surfaces extending in a direction orthogonal to the z-axis direction (in other words, extending in the x-axis direction and in the y-axis direction). The top wall 211 is joined to the end portions of the side wall 2131, the side wall 2132, and the side walls 2133 near the top surface 101. The bottom wall 212 is joined to the end portions of the side wall 2131, the side wall 2132, and the side walls 2133 near the bottom surface 102.

Accordingly, the tubular body 21 has a hollow space 210 defined by the top wall 211, the bottom wall 212, the side wall 2131, the side wall 2132, and the side walls 2133. The hollow space 210 is elongated in the z-axis direction.

The tubular body 21 has the cavity 2110 formed through the top wall 211. The hollow space 210 of the tubular body 21 communicates with the outside of the atomizing apparatus 10 through the cavity 2110 at the top surface 101.

The tubular body 21 includes a discharging portion 219 that is shaped tubularly. The discharging portion 219 has a cavity 2190 (first communication hole) with openings at opposite ends thereof in the x-axis direction. The discharging portion 219 is formed through the side wall 2132. Accordingly, the cavity 2190 of the discharging portion 219 is in communication with the hollow space 210 of the tubular body 21.

The discharging portion 219 is formed at a position away, by a predetermined distance, from the end of the tubular body 21 near the bottom wall 212 toward the top wall 211. Accordingly, the tubular body 21 has a recess 218 (first recess) at a position closer than the discharging portion 219 to the bottom wall 212.

### (Tubular Body 22)

The tubular body 22 includes a first portion 221 and a second portion 222, both of which are shaped tubularly. The first portion 221 and the second portion 222 are connected to each other, thereby forming a hollow space 220 in the tubular body 22.

The first portion 221 is elongated in the z-axis direction. A cover member 223 is connected to the end of the first portion 221 at the top surface 101. The cover member 223 has a cavity 2230 that is shaped tubularly. The cover member 223 closes the end of the first portion 221 at the top surface 101 except for the cavity 2230. Accordingly, the cavity 2230 of the cover member 223 is in communication with the hollow space 220 of the tubular body 22. Accordingly, the hollow space 220 of the tubular body 22 communicates with the outside of the atomizing apparatus 10 through the cavity 2230 of the cover member 223 at the top surface 101.

The second portion 222 is connected to the end of the first portion 221, the end being positioned near the bottom surface 102. The second portion 222 is a curved portion of which the shape is transitioned from a shape elongated in the z-axis direction to a shape elongated in the x-axis direction.

The end of the second portion 222, which is opposite to the end joined to the first portion 221, is joined to the tubular body 21 at a position near the bottom wall 212. More specifically, the end of the second portion 222, which is opposite to the end joined to the first portion 221, is joined to the tubular body 21 in such a manner that the second portion 222 surrounds the discharging portion 219 of the tubular body 21. To put it another way, the second portion 222 of the tubular body 22 is joined to the tubular body 21 in such a manner that the discharging portion 219 protrudes into the second portion 222 of the tubular body 22.

The discharging portion 219 is formed at a position away, by a predetermined distance, from the bottom of the second portion 222 (in other words, from the inside surface of the atomizing apparatus 10 near the bottom surface 102) toward the top surface 101. Accordingly, the second portion 222 has a space having a predetermined volume near the bottom surface 102 and below the discharging portion 219. This space serves as a storage space for a liquid.

The second portion 222 includes a wall portion that covers the discharging portion 219. A through-hole 2240 (second communication hole) is formed through the wall portion. The through-hole 2240 is in communication with the cavity 2190. The aperture area of the through-hole 2240 is smaller than that of the cavity 2190. The cavity 2190 of the discharging portion 219 of the tubular body 21 communicates with the hollow space 220 of the tubular body 22 through the through-hole 2240. In other words, the hollow space 210 of the tubular body 21 communicates with the hollow space 220 of the tubular body 22 through the cavity 2190 of the discharging portion 219 of the tubular body 21 and the through-hole 2240. This region corresponds to the communication region of the present disclosure.

### (Bar Member 30)

A bar member 30 has a shape elongated in the z-axis direction. The bar member 30 is disposed in the hollow space 220 of the tubular body 22. More specifically, the bar member 30 is attached to a wall portion of the second portion 222, the wall portion covering the discharging portion 219. One end 301 of the bar member 30 in the longitudinal direction is positioned closer than the discharging portion 219 to the bottom surface 102 in the z-axis direction. The other end 302 of the bar member 30 is disposed substantially at the same position of the through-hole 2240 in the z-axis direction. The other end 302 overlaps part of the through-hole 2240 as viewed in the x-axis direction.

### (Driving Circuitry Case 23)

The driving circuitry case 23 includes a top wall 231, a side wall 232, and another side wall 233. The top wall 231 has a wall surface extending in the direction orthogonal to the z-axis. The side wall 232 and the side wall 233 are elongated in the z-axis direction. The side wall 232 and the side wall 233 are spaced from each other in the x-axis direction. The top wall 231 is joined to the end portions of the side walls 232 and 233 near the top surface 101. The side wall 232 and the side wall 233 are joined to each other except for the end portions near the top surface 101. Accordingly, the driving circuitry case 23 has a hollow space 230 enclosed by the top wall 231, the side wall 232, and the side wall 233.

The driving circuitry case 23 is disposed at a position closer than the second portion 222 of the tubular body 22 to the top surface 101. The side wall 232 of the driving circuitry case 23 is in contact with the outside surface of the first portion 221 of the tubular body 22. The side wall 233 of the driving circuitry case 23 is positioned so as to be spaced from the side wall 2132 of the tubular body 21 in the x-axis direction.

The driving circuitry case 23 accommodates a substrate 60 and a battery 600. The substrate 60 has a circuit for driving the pumps 41 and 42. The substrate 60 and the battery 600 constitute a driving circuitry member.

### (Tubular Body 24)

The tubular body 24 is formed between the tubular body 21 and the driving circuitry case 23. More specifically, the tubular body 24 has a cavity 240 defined by the side wall 2132 of the tubular body 21, the side wall 233 of the driving circuitry case 23, and other side walls that connect the side wall 2132 to the side wall 233. The shapes of the tubular body 24 and the cavity 240 are elongated in the z-axis direction, and one opening of the tubular body 24 is positioned at the top surface 101 of the atomizing apparatus 10 so as to communicate with the outside. The other opening of the tubular body 24 communicates with a region of the hollow space 220 of the tubular body 22, the region being inside the second portion 222.

### (Pump 41 and Pump 42)

Each one of the pumps 41 and 42 has an enclosure. The enclosure accommodates an piezoelectric pump element having a piezoelectric element that vibrates to transport a gas. Each one of the pumps 41 and 42 has an intake hole and a discharge hole formed at opposing side surfaces of the enclosure. Each one of the pumps 41 and 42 intakes the gas through the intake hole and discharges the gas through the discharge hole by vibrating the piezoelectric element. Using the piezoelectric pump element enables the pump 41 and the pump 42 to be thin.

The pumps 41 and 42 are disposed in the hollow space 210 of the tubular body 21. More specifically, the pump 41 and the pump 42 are disposed in this order from the top surface 101 in the z-axis direction. The intake hole of the pump 41 is positioned in the cavity 2110. The discharge hole of the pump 41 is in communication with the intake hole of the pump 42. The discharge hole of the pump 42 is positioned so as to face the bottom wall 212. In other words, the pump 41 and the pump 42 are connected in series and thereby forms a single pump device. An intake hole 401 of the pump device (i.e., the intake hole of the pump 41) is positioned in the cavity 2110, and an discharge hole 402 of the pump device is positioned so as to face the bottom wall 212. The number of pumps that constitute the pump device is not limited to two but may be one, or three or more.

The discharge hole 402 of the pump device is disposed at a position closer than the discharging portion 219 to the top surface 101 in the z-axis direction. Moreover, the discharge hole 402 of the pump device is disposed at a position closer than the cavity 2190 of the discharging portion 219 to the top surface 101.

The discharge hole 402 of the pump device is positioned away from the inside surface of the side wall 2131 of the tubular body 21 in the x-axis direction.

### (Heat Conducting Member 51 and Heat Conducting Member 52)

The heat conducting members 51 and 52 are made of a material having a high thermal conductivity. The heat conducting member 51 is in contact with a surface of the pump 41, the surface facing the side wall 2132 of the tubular body 21. The heat conducting member 52 is in contact with a surface of the pump 42, the surface facing the side wall 2132 of the tubular body 21. Part of the heat conducting member 51 and part of the heat conducting member 52 protrude into the cavity 240 of the tubular body 24. For example, as illustrated in FIG. 3, the heat conducting member 52 includes a base member 521 and multiple fins 522. The base member 521 is in contact with the pump 42 and the fins 522 protrude into the cavity 240 of the tubular body 24.

### (Operation Example)

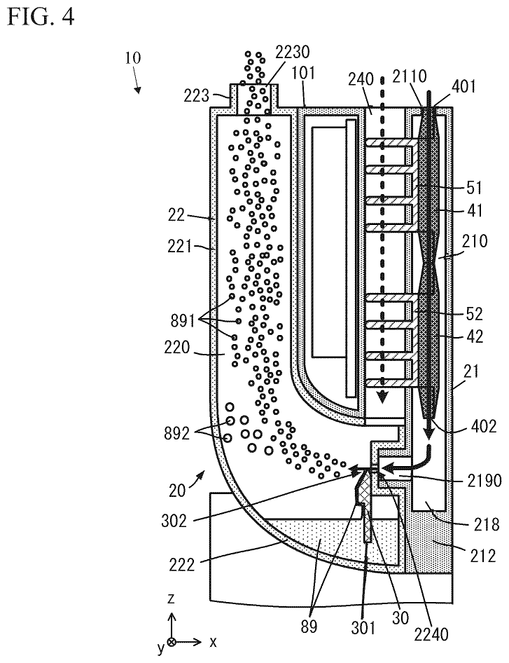

FIG. 4 is a cross-sectional side view illustrating the atomizing apparatus of the first embodiment in normal operation. FIG. 4 illustrates the same section as that illustrated in FIG. 2B.

In normal operation, the atomizing apparatus 10 is placed such that the top surface 101 faces upward and the bottom surface 102 faces downward in the vertical direction.

A liquid 89 to be atomized is stored in the second portion 222 of the tubular body 22 at the bottom surface. The liquid 89 is stored so as not to reach the discharging portion 219. Here, the one end 301 of the bar member 30 is immersed in the liquid 89.

When the pumps 41 and 42 operate in this state, the pumps 41 and 42 intake a gas through the intake hole 401 and discharge the gas through the discharge hole 402. The gas is thereby taken in the tubular body 21 through the cavity 2110 at the top surface 101 of the main body 20 and is discharged toward the bottom wall 212 of the tubular body 21.

The gas discharged from the discharge hole 402 of the pump 42 flows into the cavity 2190 of the discharging portion 219 and further flows through the through-hole 2240 into the hollow space 220 of the tubular body 22. Here, the aperture area of the through-hole 2240 is smaller than that of the cavity 2190. This increases the velocity of the gas flowing into the hollow space 220 of the tubular body 22.

While the discharged gas flows into the hollow space 220 of the tubular body 22, the gas hits the other end 302 of the bar member 30.

The one end 301 of the bar member 30 is immersed in the liquid 89, and the liquid 89 is transported along the bar member 30 to the other end 302 due to capillary action. In this state, the gas hits the other end 302 at a predetermined speed and atomizes the liquid 89. This portion of the atomizing apparatus 10 constitutes an atomizing portion. More specifically, the atomizing portion of the atomizing apparatus 10 is where the bar member 30 is disposed so as to be in contact or in proximity with the through-hole 2240. In other words, the atomizing portion is in proximity with the cavity 2190 and the through-hole 2240 through which the tubular body 21 communicates with the tubular body 22.

Atomized liquids 891 and 892 are transported inside the hollow space 220 by a gas flow from the through-hole 2240 into the hollow space 220. Here, the tubular body 22 is curved such that the second portion 222 extending in the x-axis direction is curvedly connected to the first portion 221 extending in the z-axis direction. Accordingly, the liquid 892 with large droplets hits the side wall of the first portion 221 and returns to a reservoir. On the other hand, the liquid 891 with small droplets are transported in the z-axis direction with the gas flow. The liquid 891 reaches the cavity 2230 at the top surface 101 and is discharged through the cavity 2230 to the outside.

In summary, the atomizing apparatus 10 takes the gas in at the top surface 101 and transports the gas vertically downward and then horizontally near the bottom surface of the main body 20. When the gas is transported horizontally, the atomizing apparatus 10 atomizes the liquid 89. The atomizing apparatus 10 subsequently transports the atomized liquid 891 vertically upward and discharges the atomized liquid 891 to the outside from the top surface 101 of the main body 20.

### (Specific Examples of Effects of Atomizing Apparatus 10)

The discharge hole 402 of the pump 42 is disposed at a position closer than the cavity 2190 of the discharging portion 219 to the top surface 101. With this configuration, the atomizing apparatus 10 can reduce the likelihood of the liquid 89 reaching the discharge hole 402 of the pump 42 even if the liquid 89 enters the tubular body 21 through the cavity 2190.

This reduces the likelihood of the liquid 89 entering the pumps 41 and 42. Accordingly, the atomizing apparatus 10 can reduce the likelihood of the pumps 41 and 42 malfunctioning, which improves the reliability of the atomizing apparatus 10. The liquid 89 flowing into the pumps 41 and 42 tends to cause malfunctions especially when piezoelectric pumps are used because of the presence of the electrodes or the like. The atomizing apparatus 10, however, can reduce the occurrence of malfunctions even if the pumps 41 and 42 are formed of piezoelectric pumps, which improves the reliability of the atomizing apparatus 10.

The through-hole 2240 and cavity 2190, through which the gas for atomizing the liquid 89 passes, are formed through in the x-axis direction so as to transport the gas in the x-axis direction. This reduces the likelihood of the liquid 89 staying in the through-hole 2240 and entering the tubular body 21 compared with a structure in which the through-hole 2240 and cavity 2190 are formed through in the z-axis direction so as to transport the gas toward the top surface 101.

This reduces the likelihood of the liquid 89 entering the discharge hole 402 of the pump 42 and causing malfunction of the pumps 41 and 42.

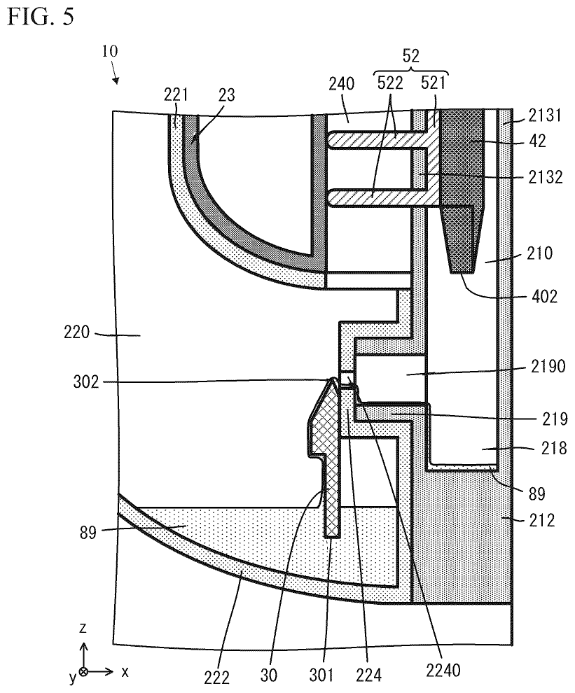

The tubular body 21 has the recess 218 at a position closer than the discharging portion 219 to the bottom wall 212. FIG. 5 is an enlarged cross-sectional view illustrating an example state in which the liquid is stored in the recess. As illustrated in FIG. 5, even if the liquid 89 enters the tubular body 21 through the cavity 2190, the liquid 89 flows into the recess 218.

Accordingly, the atomizing apparatus 10 can reduce the likelihood of the liquid 89 reaching the discharge hole 402 of the pump 42.

Note that the recess 218 can be omitted. Even in this case, the tubular body 21 can have a predetermined distance between the discharge hole 402 of the pump 42 and the inside surface of the bottom wall 212, and the liquid 89 can stay in this region of the hollow space 210. Accordingly, the atomizing apparatus 10 can reduce the likelihood of the liquid 89 reaching the discharge hole 402 of the pump 42. Providing the recess 218 can increase the volume for accommodating the liquid 89 at a position closer than the discharge hole 402 of the pump 42 to the bottom wall 212 in the tubular body 21. Accordingly, the atomizing apparatus 10 can reduce the likelihood of the liquid 89 reaching the discharge hole 402 of the pump 42 more reliably.

A reservoir for the liquid 89 is formed at a position closer than the through-hole 2240 to the bottom surface 102 of the auxiliary housing 90. Accordingly, the liquid 89 stored in the reservoir does not enter the through-hole 2240 easily.

Accordingly, the atomizing apparatus 10 can reduce the likelihood of the liquid 89 reaching the discharge hole 402 of the pump 42.

The discharging portion 219 protrudes into the hollow space 220 of the tubular body 22 from an inside surface of the second portion 222 of the tubular body 22, the inside surface being closer to the tubular body 21. With this structure, even if the atomizing apparatus 10 is overturned with the tubular body 21 facing downward, the liquid 89 stays inside the reservoir until the liquid 89 reaches the discharging portion 219 protruding into the hollow space 220 of the tubular body 22, which prevents the liquid 89 from entering the through-hole 2240 easily.

This can reduce the likelihood of the liquid 89 entering the tubular body 21 even if the atomizing apparatus 10 is overturned.

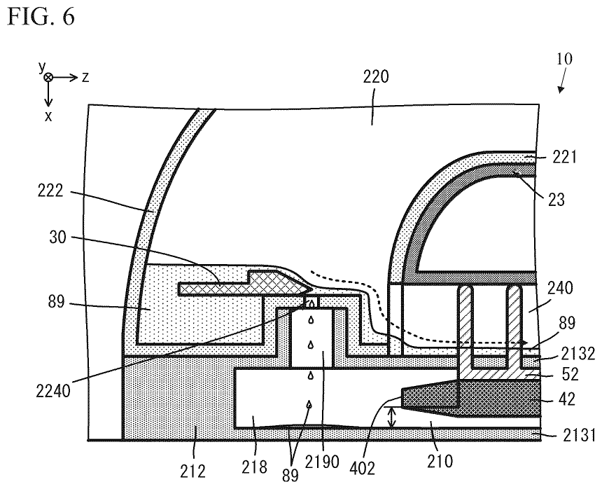

The cavity 240 of the tubular body 24 is in communication with the hollow space 220 of the tubular body 22. FIG. 6 is a view illustrating the atomizing apparatus being turned over on its side. As illustrated in FIG. 6, the aperture area of the through-hole 2240 is smaller than that of the cavity 240. In this overturned state, the temporal bottom of the tubular body 24 comes to a position lower than the end face of the discharging portion 219. Accordingly, even if the liquid 89 flows over the end face of the discharging portion 219, the liquid 89 enters the cavity 240 more easily than the through-hole 2240.

Accordingly, the atomizing apparatus 10 can reduce the likelihood of the liquid 89 entering the tubular body 21 even if the atomizing apparatus 10 is turned over. Accordingly, the atomizing apparatus 10 can reduce the likelihood of the liquid 89 reaching the discharge hole 402 of the pump 42.

The discharge hole 402 of the pump 42 is disposed at a position spaced from the side wall 2131. Accordingly, as illustrated in FIG. 6, when the atomizing apparatus 10 is turned over, the discharge hole 402 of the pump 42 comes to a position upward from the side wall 2131, which is the temporal bottom of the tubular body 21 in the overturned state.

Accordingly, even if the atomizing apparatus 10 is turned over and the liquid 89 enters the tubular body 21, the liquid 89 does not enter the discharge hole 402 of the pump 42 until the liquid 89 reaches a predetermined height as illustrated in FIG. 6. Accordingly, the atomizing apparatus 10 can reduce the likelihood of the liquid 89 reaching the discharge hole 402 of the pump 42.

The pump 41, the pump 42, the heat conducting member 51, and the heat conducting member 52 are disposed at positions closer than the discharging portion 219 to the top surface 101, whereas the reservoir for storing the liquid 89 is disposed at a position closer than the discharging portion 219 to the bottom surface 102. This structure can increase the distance from the liquid 89 stored in the reservoir to the pump 41, the pump 42, the heat conducting member 51, and the heat conducting member 52.

Accordingly, this structure can reduce the likelihood of the heat being conducted to the liquid 89 in the reservoir from the heating elements, such as the pump 41, the pump 42, the heat conducting member 51, and the heat conducting member 52. Accordingly, the atomizing apparatus 10 can reduce the likelihood of the liquid 89 in the reservoir degenerating due to the heat exposure.

The substrate 60 and the battery 600 are disposed at positions closer than the discharging portion 219 to the top surface 101, whereas the reservoir of the liquid 89 is disposed at a position closer than the discharging portion 219 to the bottom surface 102. This structure can increase the distance from the liquid 89 stored in the reservoir to the substrate 60 and the battery 600.

This can reduce the likelihood of the heat being conducted from the heating elements, such as the substrate 60 and the battery 600, to the liquid 89 in the reservoir. Accordingly, the atomizing apparatus 10 can reduce the likelihood of the liquid 89 in the reservoir degenerating due to the heat exposure.

The heating elements, such as the pump 41, the pump 42, the heat conducting member 51, the heat conducting member 52, the substrate 60, and the battery 600, are disposed at positions closer than the discharging portion 219 to the top surface 101, whereas the auxiliary housing 90 is disposed at a position closer than the discharging portion 219 to the bottom surface 102. This structure can increase the distance between the auxiliary housing 90 and the heating elements in the atomizing apparatus 10.

This can reduce the likelihood of the heat being conducted from the heating elements of the atomizing apparatus 10 to the auxiliary housing 90. In other words, this can reduce the likelihood of the auxiliary housing 90 being heated to a high temperature, which leads to a user friendly structure, for example, in the case of the auxiliary housing 90 being used as a grip for a user.

The heat conducting member 51 and the heat conducting member 52 protrude into the cavity 240. The cavity 240 is in communication with the outside at the top surface 101. The aperture area of the cavity 240 is larger than that of the through-hole 2240. The position at which the cavity 240 communicates with the hollow space 220 is closer than the through-hole 2240 to the tubular body 21. This structure enables the air flowing through the cavity 240 to cool the heat conducting member 51 and the heat conducting member 52 effectively.

Accordingly, the pumps 41 and 42 can be cooled effectively, which can reduce the likelihood of the atomizing apparatus 10 deteriorating in the atomization efficiency due to the heat generation during continuous running.

### Second Embodiment

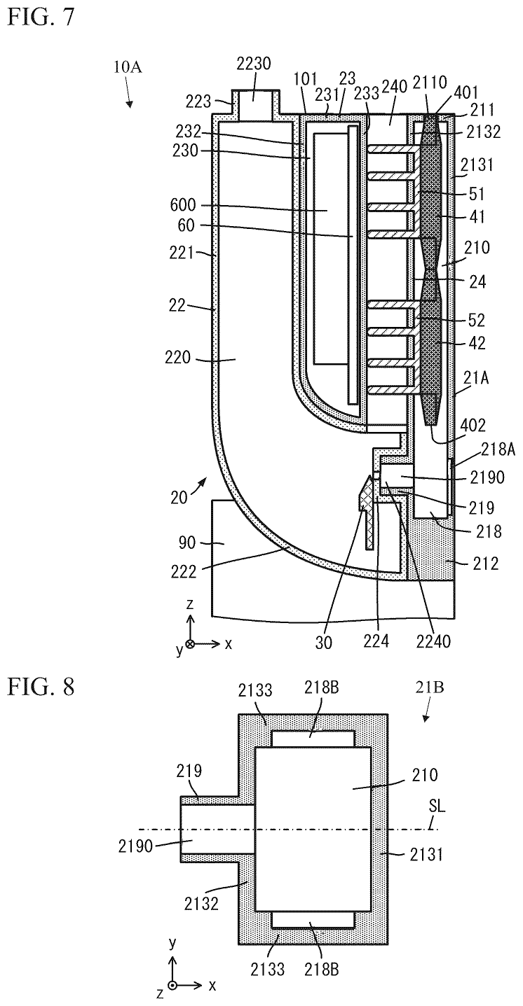

An atomizing apparatus according to a second embodiment of the present disclosure will be described with reference to the drawings. FIG. 7 is a cross-sectional side view illustrating part of a structure of the atomizing apparatus according to the second embodiment.

As illustrated in FIG. 7, an atomizing apparatus 10A of the second embodiment is different from the atomizing apparatus 10 of the first embodiment in that the atomizing apparatus 10A includes a tubular body 21A. Other configurations of the atomizing apparatus 10A are similar to those of the atomizing apparatus 10, and the same descriptions are not repeated.

The tubular body 21A has a recess 218A, which is different from the tubular body 21 of the first embodiment. Other configurations of the tubular body 21A are similar to those of the tubular body 21, and the same descriptions are not repeated.

The tubular body 21A has the recess 218A (second recess). The recess 218A is formed in the side wall 2131 so as to face the hollow space 210. The side wall 2131 opposes the side wall 2132 through which the discharging portion 219 and the cavity 2190 are formed. More specifically, the recess 218A is formed at a position, in the z-axis direction, at which the discharging portion 219 is formed.

Accordingly, when the atomizing apparatus 10A is turned over (as is the case illustrated in FIG. 6), the liquid 89 stays in the recess 218A even if the liquid 89 enters the tubular body 21A. Accordingly, the atomizing apparatus 10A can reduce the likelihood of the liquid 89 reaching the discharge hole 402 of the pump 42 more reliably.

Note that the recess is not limited to that formed in the side wall 2131, but may be configured as below. FIG. 8 is a cross-sectional view illustrating part of a structure of an atomizing apparatus according to an example derived from the second embodiment.

As illustrated in FIG. 8, a tubular body 21B has multiple recesses 218B. The recesses 218B are formed in respective side walls 2133 so as to face the hollow space 210. The side walls 2133 are the walls that connect the side wall 2131 to the side wall 2132.

Note that each one of the side walls 2133 may have the recess 218B or at least one of the side walls 2133 may have the recess 218B. It is sufficient that the recess 218B have a shape that is recessed from the wall surface in a direction intersecting a straight line (for example, line SL in FIG. 8) extending in the direction normal to the opening face of the cavity 2190 in the tubular body 21B. The tubular body having a pump may have both the recess 218A and the recess 218B.

In the above embodiments, the cavity 2190 and the through-hole 2240 are formed separately. The cavity 2190 and the through-hole 2240, however, may be formed integrally. In this case, the “first communication hole” and the “second communication hole” of the present disclosure refer to the same hole.

In the above embodiments, the cavity 2190 and through-hole 2240 extend in the second direction. However, the direction extending between the opposing openings of the cavity 2190 and through-hole 2240 may extend obliquely relative to the second direction. In other words, the direction extending between the opposing openings of the cavity 2190 and through-hole 2240 need not be parallel to the first direction or to the second direction.

For example, the end portion of the cavity 2190 and through-hole 2240 in the tubular body 22 may be disposed at a position closer to the bottom surface 102 from the end portion thereof positioned in the tubular body 21. In this case, even if the liquid 89 enters the through-hole 2240, the liquid 89 returns toward the tubular body 22. Accordingly, the atomizing apparatus can reduce the likelihood of the liquid 89 reaching the discharge hole 402 of the pump 42.

The end portion of the cavity 2190 and through-hole 2240 in the tubular body 21 may be disposed at a position closer to the bottom surface 102 from the end portion thereof positioned in the tubular body 22. In this case, the distance between the cavity 2190 and the discharge hole 402 of the pump 42 can be made larger. Accordingly, the atomizing apparatus can reduce the likelihood of the liquid 89 reaching the discharge hole 402 of the pump 42.

Note that in the above embodiments, the shapes of the hollow spaces of the tubular bodies are not limited to cuboids but may be other shapes, such as cylindrical shapes.

### Third Embodiment

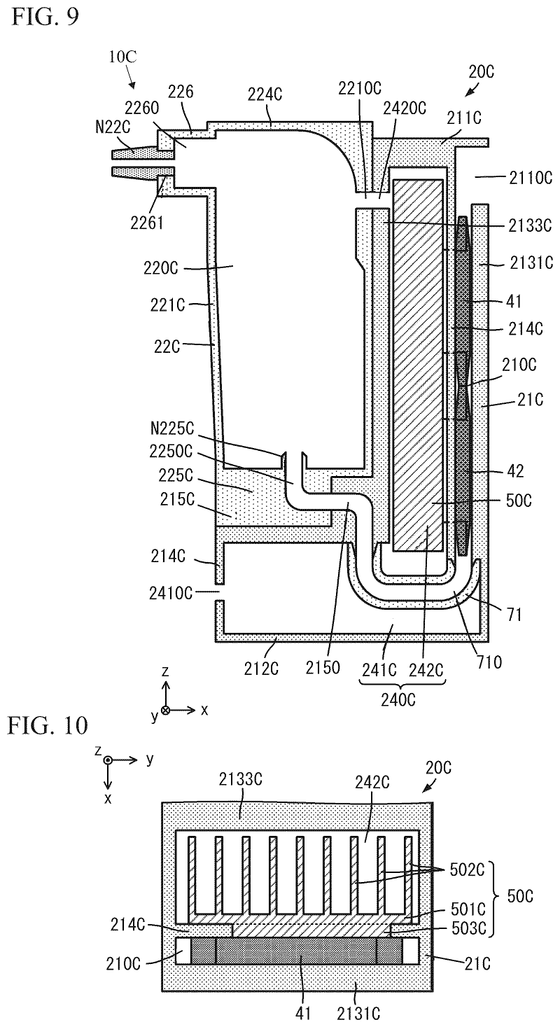

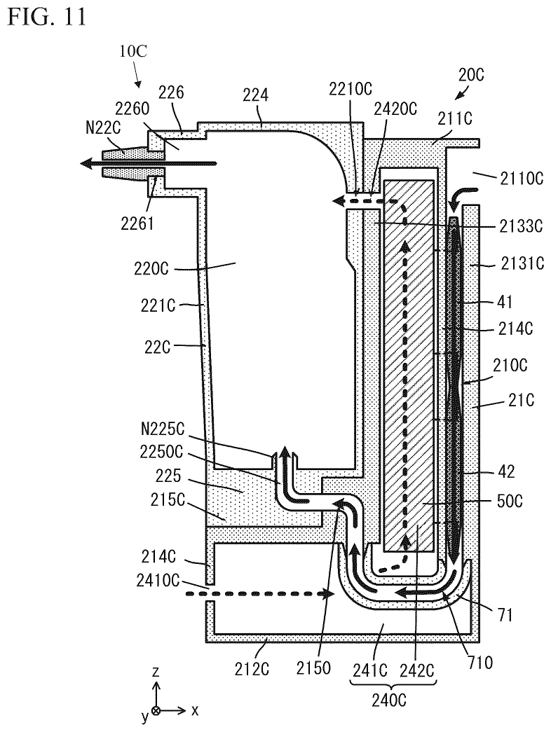

An atomizing apparatus according to a third embodiment of the present disclosure will be described with reference to the drawings. FIG. 9 is a cross-sectional side view illustrating part of a structure of the atomizing apparatus according to the third embodiment. FIG. 10 is a cross-sectional view illustrating some flow paths in the atomizing apparatus of the third embodiment when the fluid paths are viewed in plan. FIG. 11 is another cross-sectional side view schematically illustrating gas flow in the atomizing apparatus of the third embodiment.

As illustrated in FIGS. 9 and 10, the basic functional part of the atomizing apparatus 10C of the third embodiment is similar to that of the atomizing apparatus 10 of the first embodiment, and the same descriptions will not be repeated.

The atomizing apparatus 10C includes a main body 20C and an auxiliary housing (not illustrated). The main body 20C includes a tubular body 21C, a tubular body 22C, the pumps 41 and 42, a heat conducting member 50C, and a pipe 71.

The tubular body 21C includes a top wall 211C, a bottom wall 212C, a side wall 2131C, another side wall 2133C, and a partition wall 214C. The external shape of the tubular body 21C is formed by the top wall 211C, the bottom wall 212C, the side wall 2131C, the side wall 2133C, and other side walls (not illustrated) orthogonally connected to these side walls. The top wall 211C has a recess 215C recessed toward the bottom wall 212C. The side wall 2133C is the wall extending in the z-axis direction in the recess 215C.

The tubular body 21C has a hollow space 210C and a hollow space 240C. The hollow space 240C includes a hollow space 241C and a hollow space 242C.

The hollow space 210C is positioned between the side wall 2131C and the partition wall 214C. The hollow space 210C is elongated in the z-axis direction of the main body 20C. One end of the hollow space 210C in the z-axis direction communicates with the outside of the tubular body 21C through a cavity 2110C. The cavity 2110C opens near an end portion of the side wall 2131C, the end portion being connected to the top wall 211C. The other end of the hollow space 210C in the z-axis direction communicates with a hollow space 710 of the pipe 71.

A through-hole 2150 is formed through the tubular body 21C near the position where the side wall 2133C is connected to a bottom wall of the recess 215C recessed from the top wall 211C. One end of the through-hole 2150 is in communication with the hollow space 710 of the pipe 71.

The hollow space 241C is positioned between the bottom wall 212C and the bottom wall of the recess 215C recessed from the top wall 211C and extends in the x-axis direction of the main body 20C. One end of the hollow space 241C in the x-axis direction communicates with the outside of the tubular body 21C through a cavity 2410C formed through the side wall 2133C. The other end of the hollow space 241C in the x-axis direction communicates with the hollow space 242C.

The hollow space 242C is a space between the side wall 2133C and the partition wall 214C and elongated in the z-axis direction of the main body 20C. The hollow space 242C and the hollow space 210C are disposed side-by-side. One end of the hollow space 242C in the z-axis direction communicates with the hollow space 241C. The other end of the hollow space 242C in the z-axis direction communicates with the outside of the tubular body 21C through a cavity 2420C formed through the side wall 2133C.

The tubular body 22C is disposed in the recess 215C of the tubular body 21C.

The tubular body 22C includes a tubular wall 221C, a top wall 224C, and a bottom wall 225C. The external shape of the tubular body 22C is formed by the tubular wall 221C, the top wall 224C, and the bottom wall 225C. The tubular body 22C has a hollow space 220C.

The hollow space 220C communicates with the outside of the tubular body 22C through a cavity 2210C formed through the tubular wall 221C. The cavity 2210C communicates with the cavity 2420C of the tubular body 21C.

Regarding the hollow space 220C, the tubular wall 221C has a protruding portion 226 formed at a position opposing the wall portion through which the cavity 2210C is formed. A hollow space 2260 is formed in the protruding portion 226. The hollow space 2260 is in communication with the hollow space 220C. The hollow space 2260 communicates with the outside of the tubular body 22C through a cavity 2261 formed at the protruding portion 226. A nozzle N22C is fitted in the cavity 2261.

A nozzle N225C is formed at the bottom wall 225C of the hollow space 220C. The hole of the nozzle N225C is in communication with a through-hole 2250C formed through the bottom wall 225C.

The pumps 41 and 42 are disposed in the hollow space 210C. More specifically, the pump 41 and the pump 42 are disposed in this order from the cavity 2110C in the z-axis direction. The pump 41 and the pump 42 are connected in series.

The heat conducting member 50C is disposed mainly in the hollow space 242C and is in contact with the pumps 41 and 42.

More specifically, as illustrated in FIG. 10, the heat conducting member 50C includes a base member 501C, multiple fins 502C, and a contact member 503C.

The base member 501C is a tabular plate elongated in the direction parallel to the longitudinal direction of the hollow space 242C (in the direction of the row of the pumps 41 and 42 extending). The longitudinal length of the base member 501C may be greater than the longitudinal length of the pump device (the length of the pumps 41 and 42 connected in series).

The base member 501C is disposed in the hollow space 242C near the partition wall 214C. For example, the base member 501C is disposed so as to be in contact with the partition wall 214C. In this case, the base member 501C may overlap at least part of the pump device, and also may overlap the entirety of the pump device, as viewed in the x-axis direction.

The fins 502C are flat plates. The fins 502C are disposed in the hollow space 242C. The fins 502C are disposed parallel to each other so as to extend in the longitudinal direction of the hollow space 242C. The fins 502C are joined to the base member 501C in such a manner that the principal surfaces of the fins 502C extend so as to orthogonally intersect the principal surface of the base member 501C (i.e., the surfaces of the base member 501C extending in the y-axis direction). The fins 502C are disposed side-by-side at intervals in the y-axis direction (i.e., in the direction orthogonal to the longitudinal direction of the fins 502C).

The height of the fins 502C may be close to the distance between the partition wall 214C and the side wall 2133C in the hollow space 242C.

A contact member 503C is connected to a principal surface of the base member 501C that is opposite to the principal surface to which the fins 502C are connected. The connection member 503C passes through the cavity formed through the partition wall 214C and is in contact with the pumps 41 and 42.

The pumps 41 and 42 are actuated in the atomizing apparatus 10C configured as above. The atomizing apparatus 10C thereby generates a gas flow as illustrated in FIG. 11 and atomizes and discharges a liquid.

The pumps 41 and 42 in operation takes a gas into the hollow space 210C through the cavity 2110C. The gas coming from the hollow space 210C flows through the hollow space 710 of the pipe 71, the through-hole 2150, and the through-hole 2250C and reaches the nozzle N225C.

For example, the liquid 89 (not illustrated) is stored in a recess formed around the nozzle N225C that protrudes from the bottom wall 225C. The liquid 89 is supplied to the tip end of the nozzle N225C using a member similar to the bar member 30 of the above embodiments.

The liquid 89 is atomized by the gas discharged from the nozzle N225C into the hollow space 220C. The gas containing the atomized liquid 89 flows upward (toward the top wall 224C) in the hollow space 220C and is discharged out through the nozzle N22C.

Here, the flow of the gas containing the atomized liquid 89 takes a gas into the hollow space 220C through the cavity 2210C and the cavity 2420C. Accordingly, the gas is taken in from the outside of a housing 20C of the atomizing apparatus 10C through a cavity 2410C and subsequently through the hollow space 241C and the hollow space 242C. Accordingly, the atomizing apparatus 10C generates an intake gas flow passing sequentially through the cavity 2410C, the hollow space 241C, the hollow space 242C, the cavity 2420C, and the cavity 2210C.

The heat conducting member 50C, which is disposed in the hollow space 242C, is cooled effectively by this intake gas flow, which subsequently cools the pumps 41 and 42.

Accordingly, the atomizing apparatus 10C can cool the pumps 41 and 42 effectively due to the heat conducting member 50C being disposed in the hollow space 242C through which the intake gas flow passes.

Moreover, the atomizing apparatus 10C has multiple fins 502C that are disposed parallel to the flow direction of the gas. Accordingly, the fins 502C rectify the gas flow in the hollow space 242C so that the gas flows from one end thereof in communication with the hollow space 241C to the other end thereof in communication with the cavity 2420C. The flow speed of the gas increases due to the presence of multiple fins 502C compared with a case where not multiple fins 502C are present. The gas-contact area of the heat conducting member 50C becomes large. Accordingly, the heat conducting member 50C is cooled more effectively. As a result, the atomizing apparatus 10C can cool the pumps 41 and 42 more effectively.

The number of the fins 502C and intervals therebetween are not limited to what is illustrated in the drawings but can be determined appropriately in accordance with the expected effect of cooling.

In the above configuration, no element of the atomizing apparatus 10C (i.e., no obstacle) is present along a straight line connected between the nozzle N22C and the cavity 2210C along which the intake gas flows in the hollow space 220C. Accordingly, the intake gas flows fluently toward the nozzle N22C and joins the flow of the gas containing the atomized liquid 89. As a result, the atomizing apparatus 10C can increase the amount of the gas discharged from the nozzle N22C, which can improve the discharge efficiency of the liquid 89.

In addition, according to the above configuration, the direction of the gas flowing through the pumps 41 and 42 is opposite to the direction of the intake gas flowing. As a result, the atomizing apparatus 10C can further improve the cooling effect.

In the atomizing apparatus 10C, the pump 41, the pump 42, and the heat conducting member 50C are disposed at positions spaced from the bottom wall 212C although the illustration is omitted. Accordingly, when the auxiliary housing is attached to the bottom wall 212C, the auxiliary housing is disposed at a position away from the heat conducting member 50C. Accordingly, the atomizing apparatus 10C can reduce the likelihood of the auxiliary housing becoming hot.

Configurations of the above embodiments can be combined appropriately with one another, and resulted combinations are able to provide distinctive effects.

10, 10A, 10C atomizing apparatus

20, 20C main body

21, 21A, 21C 22, 22C, 24 tubular body

23 driving circuitry case

30 bar member

41, 42 pump

51, 52, 50C heat conducting member

60 substrate

71 pipe

89, 891, 892 liquid

90 auxiliary housing

101 top surface

102 bottom surface

210, 210C, 220, 230, 240C, 241C, 242C, 2260 hollow space

211, 211C top wall

212, 212C bottom wall

214C partition wall

215C recess

218, 218A recess

219 discharging portion

221 first portion

222 second portion

223 cover member

226 protruding portion

231 top wall

232 side wall

233 side wall

240 cavity

301 one end

302 other end

401 intake hole

402 discharge hole

521, 501C base member

522, 502C fins

503C contact member

600 battery

2110, 2110C, 2190, 2210C, 2230, 2420C cavity

2131, 2131C, 2132, 2133, 2133C side wall

2240, 2150, 2250C through-hole

N22C, N225C nozzle

1. An atomizing apparatus comprising:
a housing having opposing first and second ends in a first direction of the housing;
a first tubular body and a second tubular body provided in the housing;
a pump disposed in the first tubular body and having an intake hole and a discharge hole, the pump being configured to take a gas through the intake hole and discharge the gas through the discharge hole; and
an atomizing portion configured to atomize a liquid supplied to the housing by using the gas discharged through the discharge hole of the pump, wherein
the first tubular body has a first cavity and a first communication hole, the first communication hole being in communication with the second tubular body,
the second tubular body has a second cavity and a second communication hole, the second communication hole being in communication with the first communication hole,
the atomizing portion is disposed at a position adjacent to the second communication hole,
the first cavity is disposed at a position closer than the first communication hole to the first end,
in the first direction, the pump is disposed between the first communication hole and the first cavity and also between the second communication hole and the first cavity, and
in the first direction, the discharge hole of the pump is disposed at a position closer than the first communication hole and the second communication hole to the first end.

2. The atomizing apparatus according to claim 1, wherein
the first tubular body and the second tubular body are disposed side-by-side in a second direction orthogonally intersecting the first direction, and
the first communication hole and the second communication hole are elongated in a direction not orthogonal to the second direction.

3. The atomizing apparatus according to claim 2, wherein
the first communication hole and the second communication hole are elongated in the second direction.

4. The atomizing apparatus according to claim 2, wherein
the first communication hole or the second communication hole has one end communicating with a hollow space of the first tubular body and another end communicating with a hollow space of the second tubular body, and
the other end is disposed at a position closer than the one end to the second end.

5. The atomizing apparatus according to claim 2, wherein
the first communication hole or the second communication hole has one end communicating with a hollow space of the first tubular body and another end communicating with a hollow space of the second tubular body, and
the one end is disposed at a position closer than the other end to the second end.

6. The atomizing apparatus according to claim 1, wherein
the first tubular body has a first recess recessed from the first communication hole toward the second end.

7. The atomizing apparatus according to claim 1, wherein
the first tubular body has a second recess recessed from an inside surface thereof at a position opposing the first communication hole.

8. The atomizing apparatus according to claim 1, wherein
the first tubular body has a second recess recessed from an inside surface of the first tubular body in a direction intersecting a straight line drawn so as to extend in a direction normal to an opening face of the first communication hole.

9. The atomizing apparatus according to claim 1, further comprising:
a reservoir disposed at a position closer than the second communication hole to the second end and configured to store the liquid.

10. The atomizing apparatus according to claim 9, wherein
the reservoir is provided in the second tubular body at a position closer than the second communication hole to the second end, and
a wall surface of the reservoir near the first tubular body is disposed at a position closer than the second communication hole to the first tubular body when viewed in a cross section.

11. The atomizing apparatus according to claim 10, further comprising:
a third cavity at a position between the first tubular body and the second tubular body,
the third cavity is in communication with the second tubular body, and
at least a part of a communication region between the third cavity and the second tubular body is disposed at a position closer to the first tubular body relative to a face of the second communication hole facing the second tubular body.

12. The atomizing apparatus according to claim 11, further comprising:
a heat conducting member attached to the pump, wherein
at least a part of the heat conducting member protrudes into the third cavity.

13. The atomizing apparatus according to claim 1, further comprising:
an auxiliary housing disposed at a position closer than the first communication hole and the second communication hole to the second end.

14. The atomizing apparatus according to claim 13, further comprising:
a driving circuitry member configured to drive the pump, wherein
the driving circuitry member is disposed at a position opposite to the auxiliary housing with respect to the first communication hole and the second communication hole.

15. The atomizing apparatus according to claim 2, wherein
the first tubular body has a first recess recessed from the first communication hole toward the second end.

16. The atomizing apparatus according to claim 3, wherein
the first tubular body has a first recess recessed from the first communication hole toward the second end.

17. The atomizing apparatus according to claim 4, wherein
the first tubular body has a first recess recessed from the first communication hole toward the second end.

18. The atomizing apparatus according to claim 5, wherein
the first tubular body has a first recess recessed from the first communication hole toward the second end.

19. The atomizing apparatus according to claim 2, wherein
the first tubular body has a second recess recessed from an inside surface thereof at a position opposing the first communication hole.

20. The atomizing apparatus according to claim 3, wherein
the first tubular body has a second recess recessed from an inside surface thereof at a position opposing the first communication hole.
